(19) United States
(12) Patent Application Publication
(10) Pub. No.: US 2011/0237872 A1
(43) Pub. Date: Sep. 29, 2011

(54) DYNAMIC HEART HARNESS

(75) Inventors: Samuel M. Shaolian, Newport Beach, CA (US); Ross Tsukashima, San Diego, CA (US); Scott L. Pool, Laguna Hills, CA (US)

(73) Assignee: MICARDIA CORPORATION, Irvine, CA (US)

(21) Appl. No.: 13/063,736

(22) PCT Filed: Sep. 24, 2009
(86) PCT No.: PCT/US2009/058204

§ 371 Date: Mar. 11, 2011

Related U.S. Application Data
(60) Provisional application No. 61/099,836, filed on Sep. 24, 2008.

(51) Int. Cl. A61F 2/00

(52) U.S. Cl. 600/37

(57) ABSTRACT

A reversibly adjustable heart harness is configured to surround at least a portion of a heart and to provide a compressive force to the heart during at least a portion of a cardiac cycle. The heart harness includes a plurality of wires forming a mesh structure, and one or more tensioning motors connected to the mesh structure. The one or more tensioning motors are configured to selectively increase or reduce tension in the mesh structure to readjust the compressive force provided that the heart harness provides to the heart.

### TECHNICAL FIELD

This application is related to systems and methods for treating a heart. More specifically, this application is related to reversibly adjustable harnesses configured to fit around at least a portion of a heart.

### BACKGROUND

Congestive heart failure (“CHF”) is characterized by the failure of the heart to pump blood at sufficient flow rates to meet the metabolic demand of tissues, especially the demand for oxygen. One characteristic of CHF is remodeling of portions of a patient's heart. Remodeling involves physical change to the size, shape, and/or thickness of the heart wall. For example, a damaged left ventricle may have some localized thinning and stretching of a portion of the myocardium. The thinned portion of the myocardium often is functionally impaired, and other portions of the myocardium attempt to compensate. As a result, the other portions of the myocardium may expand so that the stroke volume of the ventricle is maintained notwithstanding the impaired zone of the myocardium. Such expansion may cause the left ventricle to assume a somewhat spherical shape.

Cardiac remodeling often subjects the heart wall to increased wall tension or stress, which further impairs the heart's functional performance. Often, the heart wall will dilate further in order to compensate for the impairment caused by such increased stress. Thus, a cycle can result in which dilation leads to further dilation and greater functional impairment.

Historically, congestive heart failure has been managed with a variety of drugs. Devices have also been used to improve cardiac output. For example, left ventricular assist pumps help the heart to pump blood. Various skeletal muscles, such as the latissimus dorsi, have been used to assist ventricular pumping. Researchers and cardiac surgeons have also experimented with prosthetic “girdles” disposed around the heart. One such design is a prosthetic “sock” or “jacket” that is wrapped around the heart. The proper degree of tension provided by a prosthetic jacket, however, is difficult to determine during heart surgery. This is due to the fact that the patient is under general anesthesia, in a prone position, and with the chest wide open. These factors affect the normal operation of the heart muscle. Even if the synching is done well, the tissue may continue to relax over the patient's lifetime such that the heart condition returns.

### BRIEF DESCRIPTION OF THE DRAWINGS

FIG. 1 schematically illustrates a heart harness covering a portion of a heart according to one embodiment.

FIG. 2 is a schematic diagram of a magnetic tensioning motor according to one embodiment.

FIG. 3A is a schematic diagram of a front view of a magnet according to one embodiment.

FIG. 3B is a schematic diagram of a side view of the magnet shown in FIG. 3A according to one embodiment.

FIG. 4 schematically illustrates a side view of an external magnet used to rotate the magnets of in a heart harness according to one embodiment.

FIG. 5 is a schematic diagram of an adjustment device that includes two magnets arranged outside of a patient's body according to one embodiment.

FIG. 6 is a simplified block diagram of a system for adjusting the tension of a heart harness according to one embodiment.

FIGS. 7A, 7B, 7C, and 7D schematically illustrate a method for manufacturing a mesh structure of a heart harness according to one embodiment.

FIGS. 8A, 8B, and 8C illustrate an alternate configuration for a link according to one embodiment.

FIGS. 9A, 9B, and 9C illustrate another alternate configuration according to one embodiment.

FIG. 10 schematically illustrates a mesh structure of a heart harness having a plurality of parallel tensioning motors according to one embodiment.

FIG. 11 schematically illustrates a mesh structure of a heart harness having a plurality of tensioning motors according to another embodiment.

FIG. 12 schematically illustrates a first heart harness, a second heart harness, and a third heart harness, each forming an individual mesh structure row around a heart according to one embodiment.

FIGS. 13A and 13B schematically illustrate a tensioning motor comprising “stacked” magnetostrictive elements attached between portions of a rigid or semi-rigid frame according to one embodiment.

FIG. 14 schematically illustrates a tensioning motor having ten magnetostrictive elements according to one embodiment.

FIG. 15 schematically illustrates a heart harness that uses the tensioning motor with magnetostrictive elements shown in FIG. 14 according to one embodiment.

FIGS. 16A and 16B schematically illustrate a tensioning motor that includes a magnetostrictive element in cooperation with a pulley system to adjust the tension of the mesh structure of a heart harness according to one embodiment.

### DETAILED DESCRIPTION

A reversibly adjustable heart harness according to one embodiment provides reinforcement to a heart and allows for the proper degree of tension both during heart surgery and over the patient's lifetime. In one embodiment, the heart harness may be adjusted low-invasively or non-invasively with the patient alert and postoperatively healed. In addition, the heart harness incorporates the ability to tighten and/or relax different portions of the harness with fine position control. In certain embodiments, the heart harness is configured to contract and expand in synchronization with the beating of the heart.

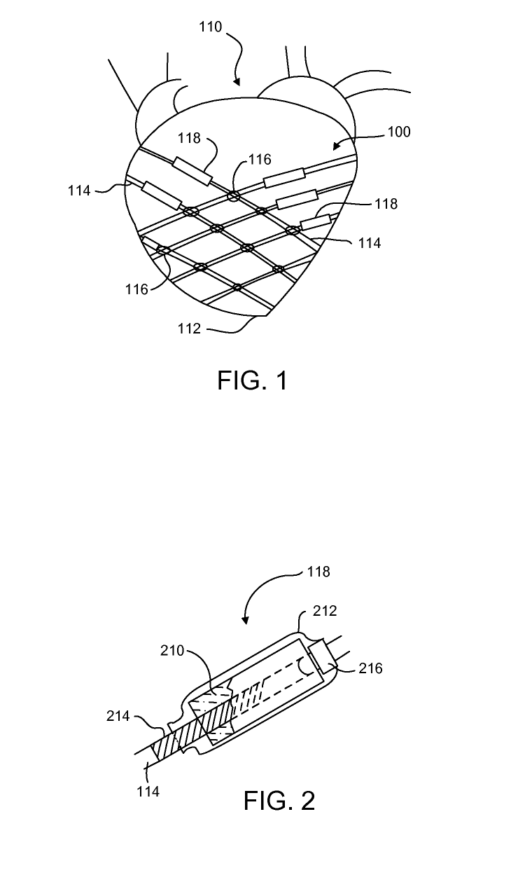

FIG. 1 schematically illustrates a heart harness 100 covering a portion of a heart 110 (e.g., a human heart or other mammalian heart) according to one embodiment. The heart harness 100 in this example covers the apex 112 and other portions (e.g., left and right ventricles) of the heart 110. The heart harness 100 provides a compressive force on the heart 110 during at least a portion of the cardiac cycle.

The heart harness 100 includes one or more wires 114 having a series of biasing elements or links 116 between wire segments that form a net or mesh structure. The links 116 deform as the heart 110 expands during filling. The heart harness 100 also includes one or more motors 118 to adjust the tension between the wires 114 and the links 116. The tensioning motors 118 may be used to fit the mesh structure of the heart harness 100 to a particular patient's heart 110 and/or to readjust the compressive forces provided by the heart harness 100 as the patient's heart 110 changes shape over time. As discussed below, in certain embodiments, the tensioning motors 118 may also be used to contract and expand the heart harness 100 in synchronization with the beating of the heart 100.

In one embodiment, the tensioning motor 118 is a magnetic motor configured to rotate in the presence of a rotating magnetic field. For example, FIG. 2 is a schematic diagram of a magnetic tensioning motor 118 according to one embodiment. The magnetic tensioning motor 118 includes a permanent magnet 210 configured to rotate within a magnet housing 212.

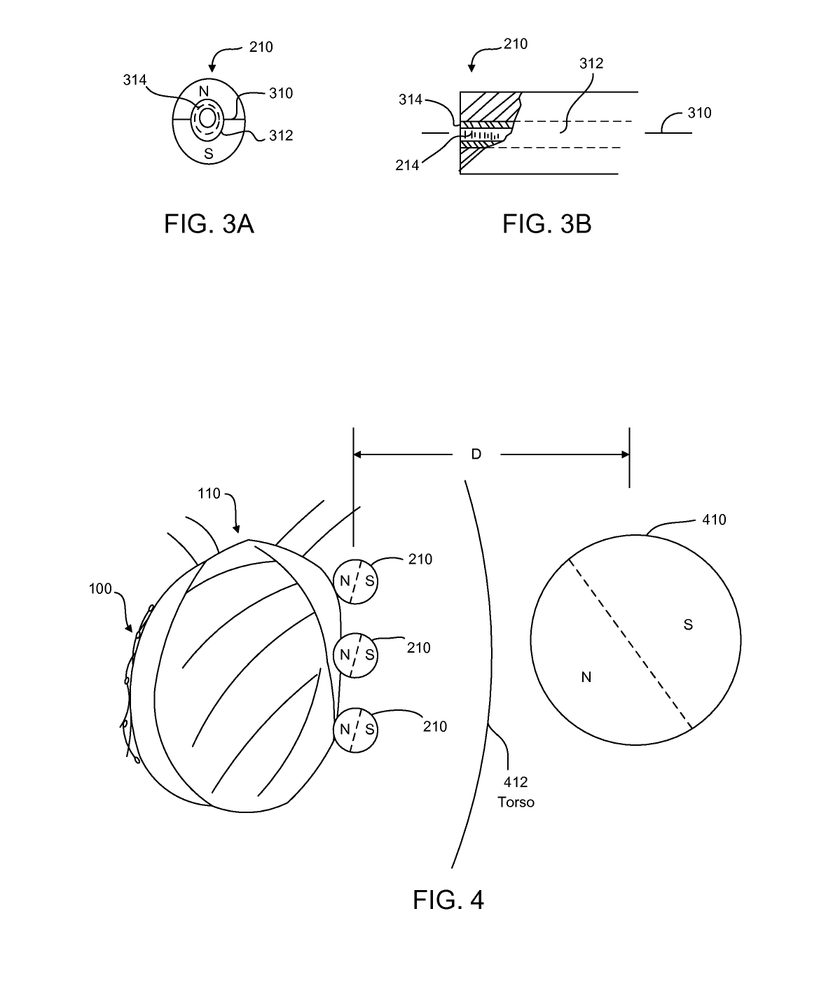

The magnet 210 is cylindrical and is configured to rotate around its cylindrical axis when exposed to a rotating magnetic field. FIG. 3A is a schematic diagram of a front view of the magnet 210 and FIG. 3B is a schematic diagram of a side view of the magnet 210. The magnet 210 has magnetic poles (e.g., north “N” and south “S”) divided along a plane 310 that runs the length of the cylinder. The magnet 210 may include a rare earth magnet and may be plated (e.g., with nickel or gold) and/or suitably encapsulated to prevent harm to the patient and damage to the magnet 210. The magnet 210 includes a hollow region 312 running along the length of the cylinder between the N and S poles. The hollow region 312 may be threaded or may contain a threaded insert 314 through which a lead screw 214 is pulled into and out of the magnet 210 as the magnet 210 turns. In another embodiment, a separate lead screw 214 is not used. Rather, threads are formed or cut into an end of the wire 114 such that the wire 114 interfaces directly with the threads in the magnet 210 (e.g., the threaded insert 314).

The magnet housing 212 may include, for example, stainless steel or another biocompatible material. The wire 114 may also include, for example, stainless steel or another biocompatible material. Although not shown, in some embodiments, the magnet housing 212 and/or the heart harness 100 may be covered with a polymeric sleeve formed from any of a variety of synthetic polymeric materials, or combinations thereof, including PTFE, PE, PET, Urethane, Dacron, nylon, polyester, or woven materials. Other component materials are also selected to provide long term contact with human or animal tissue.

In one embodiment, the heart harness 100 includes ball bearings 216 to anchor the spinning magnet 210. When the magnet 210 is exposed to a rotating magnetic field in one direction, the magnet 210 pulls the lead screw 214 and/or threaded wire 114 into the magnet 210, which in turn increases the tension on at least a portion of the mesh structure of the heart harness 100. When the magnet 210 is exposed to the magnetic field rotating in the opposite direction, the magnet 210 pushes the lead screw 214 and/or threaded wire 114 out of the magnet 210, which in turn reduces the tension on at least a portion of the mesh structure of the heart harness 100.

The tensioning motors 118 of the heart harness 100 may be controlled remotely by one or more magnets located internal or external to the patient's body. For example, FIG. 4 schematically illustrates a side view of an external magnet 410 used to rotate the magnets 210 of the heart harness 100 implanted around at least a portion of the patient's heart 110. The magnet 410 may be located external to the patient's torso 412 at a distance D from the tensioning magnets 210. Rotating the external magnet 410 rotates its magnetic field, which is coupled through the distance D to the magnetic field of the tensioning magnets 210. Thus, the magnetic fields of the respective magnets 210, 410 interact with each other such that mechanically rotating the magnet 410 (e.g., using a stepper motor) causes the magnets 210 in the heart harness 100 to rotate. For example, rotating the magnet 410 in a clockwise direction around its cylindrical axis causes the magnets 210 to rotate in a counterclockwise direction. Similarly, rotating the magnet 410 in a counterclockwise direction around its cylindrical axis causes the magnets 210 to rotate in a clockwise direction. Thus, rotating the external magnet 410 in one direction causes the tension of the heart harness 100 to increase while turning the magnet 410 in the opposite direction causes the tension of the heart harness 100 to decrease. In one embodiment, the external magnet 410 has a diameter of approximately 4 inches and may be driven by a stepper motor, as discussed below, for precise rotational control of the magnets 210 of the heart harness 100 from outside the patient's body.

The external magnet 410 provides accurate one-to-one control of the tensioning magnets 210 in the heart harness 100, assuming sufficient magnetic interaction between the magnets 210, 410. In other words, one complete rotation of the external magnet 410 will cause one complete rotation of the magnets 210 in the heart harness 100. If the relationship between the number of rotations of the magnets 210 and the tension of the heart harness 100 is linear, the tension of the heart harness 100 may be determined directly from the number of revolutions since the heart harness 100 was at its last known tension. If, however, the relationship between the number of revolutions and tension is not linear, a look-up table based on tested values for a particular harness or type of harness may be used to relate the number of revolutions to the tension of the heart harness 100. Imaging techniques may also be used to determine the resulting shape of the heart harness after adjusting the tension. In addition, or in other embodiments, the heart harness 100 may include circuitry for counting the number of revolutions of the respective tensioning magnets 210, and for communicating this data to a user. For example, the heart harness 100 may include a radio frequency identification (RF ID) tag technology to power and receive data from the heart harness 100.

While placing the magnets 210, 410 in parallel increases rotational torque on the magnets 210 in the heart harness 100, the disclosure herein is not so limited. For example, the rotational axis of the external magnet 410 may be placed at an angle θ with respect to the rotational axis of the tensioning magnet 210. The rotational torque on the magnet 210 provided by rotating the magnet 410 increases as the angle θ approaches zero degrees, and decreases as the angle θ approaches 90 degrees (assuming both magnets 210, 410 are in the same geometric plane or in parallel planes).

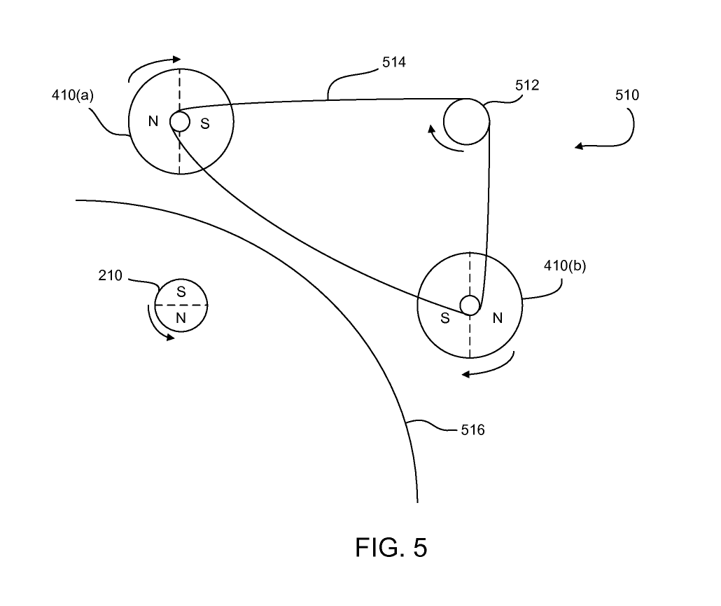

The rotational torque on the magnet 210 in the heart harness 100 also increases by using magnets 210, 410 with stronger magnetic fields and/or by increasing the number of magnets 410 used in an adjustment device. For example, FIG. 5 is a schematic diagram of an adjustment device 510 that includes two magnets 410(a), 410(b) arranged outside of a patient's body 516 according to one embodiment. An artisan will recognize from the disclosure herein that the adjustment device 510 is not limited to one or two magnets, but may include any number of magnets. The magnets 410(a), 410(b) are oriented and rotated relative to each other such that their magnetic fields add together at the tensioning magnet 210 to increase rotational torque. A computer controlled motor 512 synchronously rotates the external magnets 410(a), 410(b) through a mechanical linkage 514 to magnetically rotate the tensioning magnet 210 and adjust the tension of the heart harness 100. One revolution of the motor 512 causes one revolution of the external magnets 410(a), 410(b), which in turn causes one revolution of the tensioning magnet 210. As discussed above, by counting motor revolutions, the tension of the heart harness 100 may be calculated. In one embodiment, the motor 512 includes a gear box with a known gear ratio such that multiple motor revolutions may be counted for one magnet revolution.

In another embodiment, a strong electro-magnetic field like that used in Magnetic Resonance Imaging (MRI) is used to adjust the tension of the heart harness 100. The magnetic field may be rotated either mechanically or electronically to cause the tensioning magnet 210 in the heart harness 100 to rotate. The patient's body may also be rotated about the axis of the magnet 210 in the presence of a strong magnetic field, like that of an MRI. In such an embodiment, the strong magnetic field will hold the magnet 210 stationary while the heart harness 100 and patient are rotated around the fixed magnet 210 to cause adjustment. The tension may be determined by counting the number of revolutions of the magnetic field, or the patient's body, similar to counting revolutions of the permanent magnets 410 discussed above.

In another embodiment, the heart harness 100 may be adjusted during heart surgery. For example, after implanting the heart harness 100 around the heart 110, regurgitation may be monitored (e.g., using ultrasound color Doppler). Then, a user (e.g., surgeon) may use a handheld adjustment device 510 to adjust the tension of the heart harness 100 based on the detected regurgitation. Additional regurgitation monitoring and tension adjustment may be performed before completing the surgery.

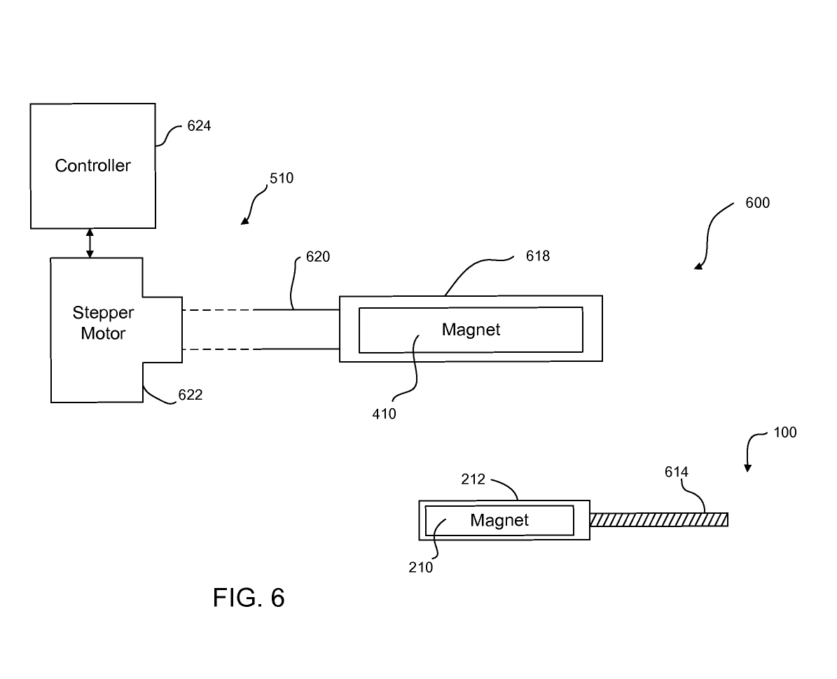

FIG. 6 is a simplified block diagram of a system 600 for adjusting the tension of the heart harness 100 according to one embodiment. The simplified embodiment shown in FIG. 6 is provided to illustrate the basic operation of the tensioning motor 118. However, more detailed embodiments are provided below.

The system 600 includes an adjustable heart harness 100 and an adjustment device 510. The heart harness 100 includes a magnet 210 in a magnet housing 212. The magnet 210 is cylindrical and is configured to rotate around its cylindrical axis when exposed to a rotating magnetic field. The magnet 210 is coupled to a proximal end of a lead screw 214 (or, in certain embodiments, a threaded end of a wire 114 within the mesh structure of the heart harness 100). The magnet 210 may include a rare earth magnet and may be plated (e.g., with nickel or gold) and/or suitably encapsulated to prevent harm to the patient and damage to the magnet 210. Other component materials are also selected to provide long term contact with human tissue. The heart harness 100 may be covered with a Dacron fabric or other suturable material.

The adjustment device 510 includes a magnet 410 in a magnet housing 618 coupled to a drive shaft 620. The drive shaft 620 may be connected to a stepper motor 622 coupled to a controller 624. The controller 624 may include, for example, a microprocessor or personal computer. The controller 624 is configured to control the position, rotation direction, rotation speed, speed ramp up/down, and other parameters of the stepper motor 622. The stepper motor 622 rotates the shaft 620, which in turn rotates the magnet 410. In certain embodiments the shaft 620 and the magnet 410 may be covered with a protective material (e.g., plating).

In operation, the rotating magnet 410 in the adjustment device 510 causes the magnet 210 in the heart harness 100 to rotate. The rotating magnet 210 moves the lead screw 614 into or out of the magnet housing 212 to either increase or decrease the tension of the heart harness 100.

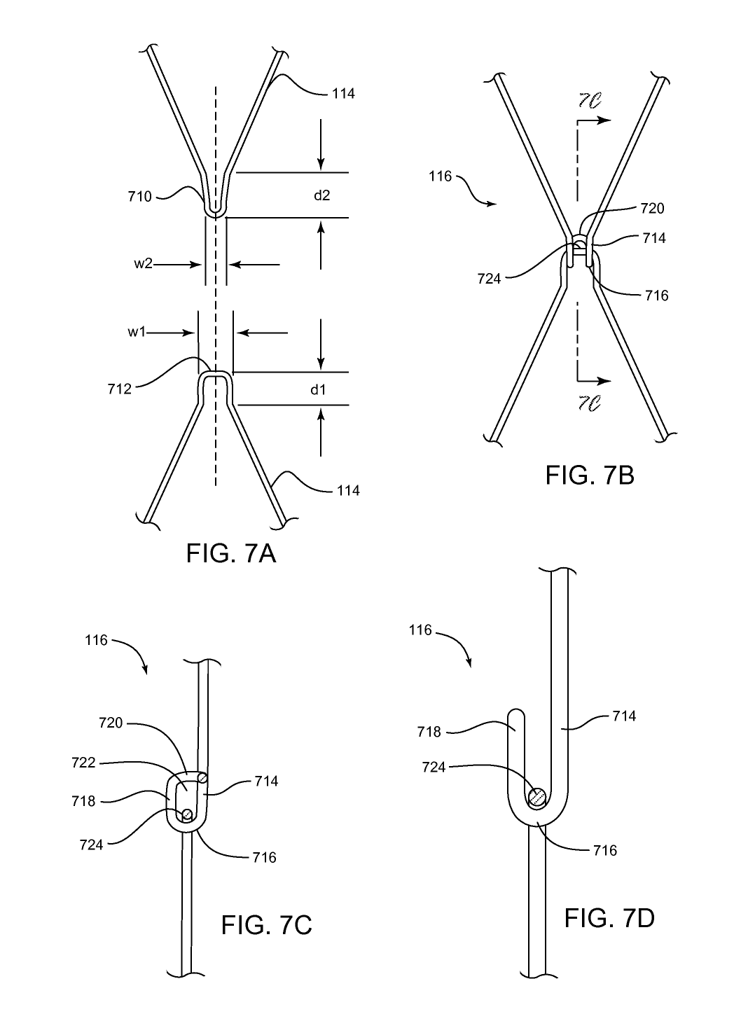

FIGS. 7A, 7B, and 7C schematically illustrate a method for manufacturing the mesh structure of the heart harness 100 according to one embodiment. As shown in FIG. 7A, the wire 114 includes apexes 710 with an elongated axial length d2, which permits the apex 710 to be wrapped around a corresponding portion 712, such as an apex of the adjacent segment, to provide an interlocking link 116 between two axially adjacent wire segments. One embodiment of the link 116 produced by the opposing apexes 710 and 712 utilizes wire 114 having a diameter in a range between approximately 0.012 inches and approximately 0.018 inches, d1 is generally within a range between approximately 1 mm and approximately 4 mm, and d2 is within a range between approximately 5 mm and approximately 9 mm. In general, a longer d2 dimension permits accommodation for greater travel of the apex 712 with respect to the apex 710, thereby permitting greater flexibility of the heart harness 100. A width W1 is within a range between approximately 3 mm and approximately 5 mm, and a width W2 is sufficiently less than W1 such that the apex 710 fits within the apex 712. Any of a wide variety of specific apex configurations and dimensions can be utilized, as will be apparent to those of skill in the art in view of the disclosure herein. Regardless of the specific dimensions, the end of the apex 710 is advanced through the apex 712, and folded back upon its self to hook the apex 712 therein to provide a link 116 in accordance with the embodiments disclosed herein.

The resulting link 116 (see FIGS. 7B and 7C) includes a wall portion 714 extending in a first direction, and a transverse portion 716 extending transverse to the first direction. A return portion 718 extends generally in the opposite direction from the wall portion 714 to create a generally “U” shaped hook. In certain embodiments, a closing portion 720 is also provided, to minimize the risk of excessive vertical compression of the heart harness 100. The forgoing structure produces a functionally closed aperture 722, which receives an interlocking section 724 of the adjacent wire segment. For an alternative embodiment, see FIG. 7D.

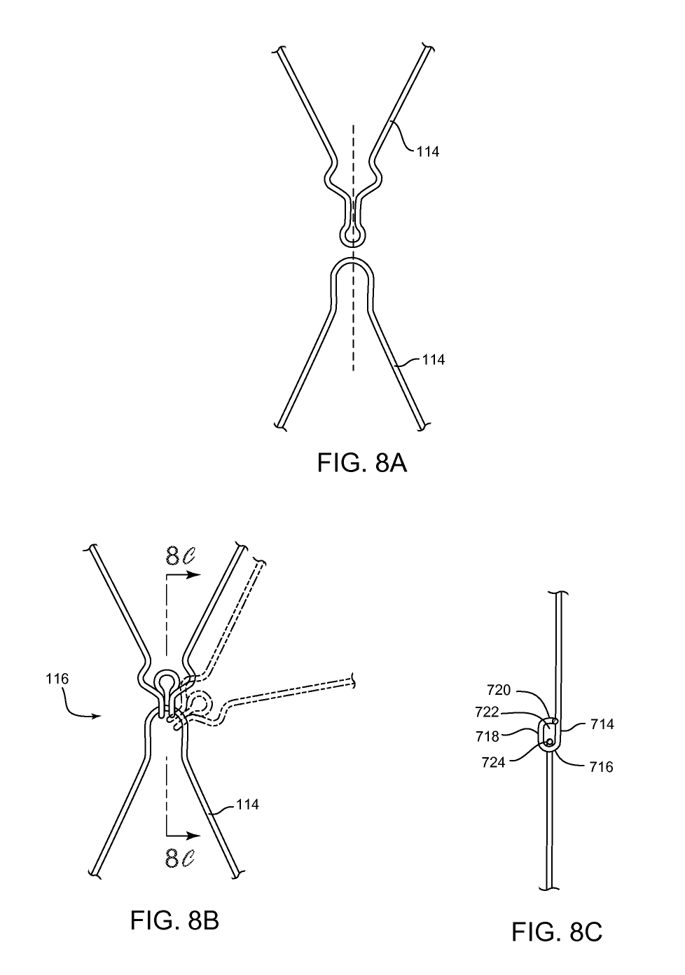

FIGS. 8A, 8B, and 8C illustrate an alternate configuration for the link 116 according to one embodiment. With this configuration, the radial expansion force may be higher than that of the configuration shown in FIGS. 7A, 7B, and 7C.

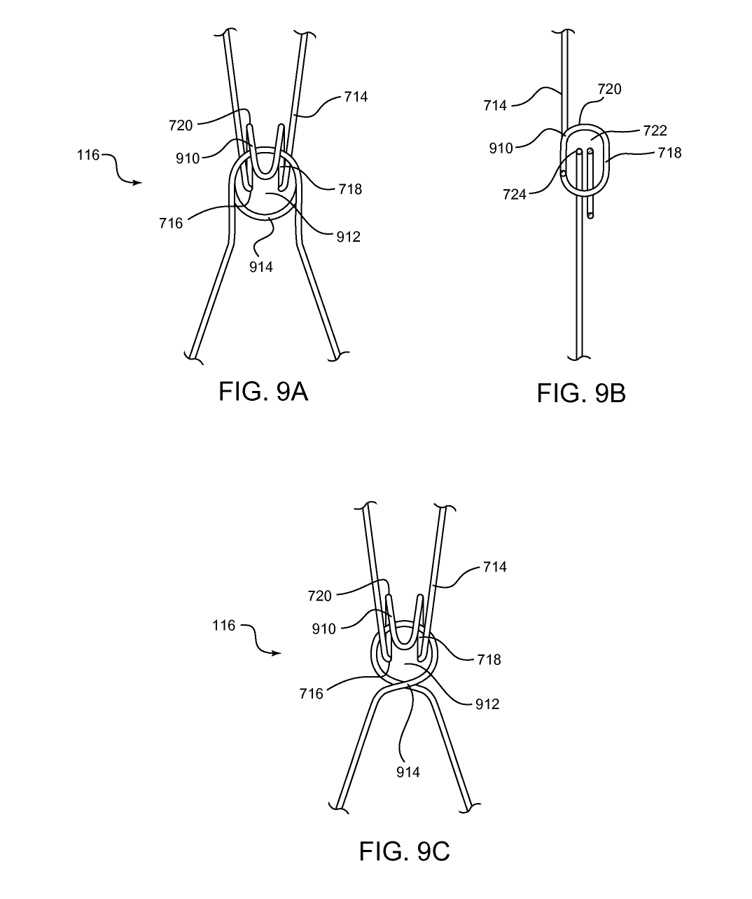

FIGS. 9A and 9B illustrate another alternate configuration according to one embodiment. This linkage 116 has a better resistance to axial compression and disengagement than that of the embodiments discussed above. In this embodiment, the apex extends beyond closing portion 720 and into an axial portion 910. Provision of an axial extension 910 provides a more secure enclosure for the aperture 722 as will be apparent to those of skill in the art. The embodiments of FIGS. 9A and 9B also illustrate an enclosed aperture 912 on the opposing apex. The aperture 912 is formed by wrapping the apex in at least one complete revolution so that a generally circumferentially extending portion 914 is provided. The circumferential portion 914 provides a stop, to limit vertical compressibility of the heart harness 100. The closed aperture 912 can be formed by winding the wire of the apex about a mandrel either in the direction illustrated in FIG. 9A, or the direction illustrated in FIG. 9C. The embodiment of FIG. 9C advantageously provides only a single wire thickness through the aperture 722, thereby minimizing the wall thickness of the heart harness 100. This is accomplished by moving the crossover point outside of the aperture 722, as will be apparent from FIG. 9C.

The link 116 in accordance with one embodiment is formed integrally with the wire 114 that forms the mesh structure of the heart harness 100. Alternatively, the link 116 may be constructed from a separate material which is secured to the mesh structure such as by soldering, suture, wrapping or the like.

An artisan will understand from the disclosure herein that not every intersection of apexes 76, 78 in the mesh structure may include a link 116, and/or that different types of links may be used at different apex intersections. The distribution of the links 116 may also be varied along the length and/or width of the mesh structure. For example, a first zone and a second zone may be provided with a relatively larger number of links 116 than a third zone in the mesh structure. The interlocking links 116 discussed herein may be utilized as the sole means of securing adjacent segments to each other, or may be supplemented by additional attachment structures such as metal loops, sutures, welds, and/or other attachment mechanisms.

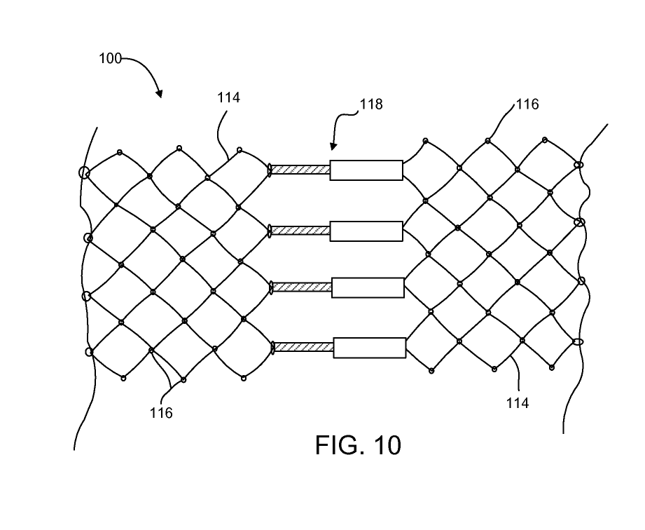

The configuration of the tensioning motors 118 within the mesh structure of the heart harness 100 may vary from that shown in FIG. 1. For example, FIG. 10 schematically illustrates a mesh structure of a heart harness 100 having a plurality of parallel tensioning motors 118 according to one embodiment. As discussed above, the mesh structure is formed by one or more linked wires 114. The mesh structure is configured to wrap around and conform to the curvature of at least a portion of the heart. In this example, the plurality of parallel tensioning motors is oriented horizontally for circumferential adjustment of the mesh structure.

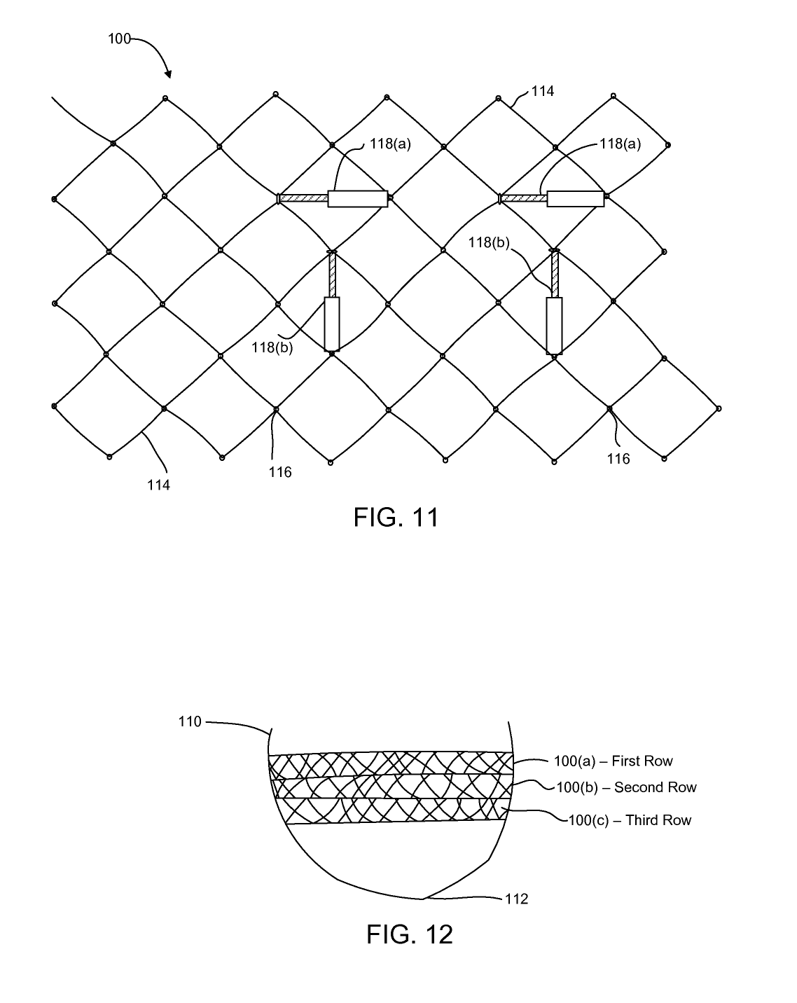

FIG. 11 schematically illustrates a mesh structure of a heart harness 100 having a plurality of tensioning motors 118 according to another embodiment. In this example embodiment, two of the tensioning motors 118(a) are oriented horizontally to provide circumferential adjustment of the heart harness 100, and two of the tensioning motors 118(b) are oriented vertically to provide height adjustment of the heart harness 100. Using tensioning motors 118(a), 118(b) oriented in different dimensions helps conform the mesh structure to the heart. An artisan will understand from the disclosure herein that other orientations may also be possible. For example, one or more tensioning motors 118 may be angled with respect to the horizontal and vertical orientations shown in FIG. 11. An artisan will also understand from the disclosure herein that any number of tensioning motors 118, in any combination of orientations, may also be used.

Further, the heart harness 100 shown in FIG. 11 may be combined with other heart harnesses 100 having various motor configurations. For example, FIG. 12 schematically illustrates a first heart harness 100(a), a second heart harness 100(b), and a third heart harness 100(c), each forming an individual mesh structure row around a heart 110 according to one embodiment. The individual heart harnesses 100(a), 100(b), 100(c) may or may not be connected with one another, depending on the particular application. Although not shown in FIG. 12, each individual heart harnesses 100(a), 100(b), 100(c) may have its own distinct configuration and orientation of the tensioning motors 118.

In other embodiments, one or more of the tensioning motors 118 shown in FIG. 1 do not include a rotating magnet 210. In one such embodiment, a tensioning motor 118 includes one or more magnetostrictive elements that changes its shape when subjected to a magnetic field. Thus, turning on and off a magnetic field, or rotating a magnetic field, contracts and/or expands the length of the magnetostrictive elements to adjust the tension in the mesh structure of the heart harness 100.

In one embodiment, the magnetostrictive element comprises Terfenol-D® available from Etrema Products, Inc. of Ames, Iowa. Terfenol-D® is a near single crystal metal alloy, which converts electrical power to mechanical power, and vice versa. Terfenol-D® is considered a “giant” magnetostrictive material that can change by approximately 1700 parts-per-million (ppm), depending on the applied magnetic field strength. When used appropriately, Terfenol-D® has the following properties: high strain, high force, wide bandwidth, “unlimited” or high cycle life, wide temperature range, and microsecond response time.

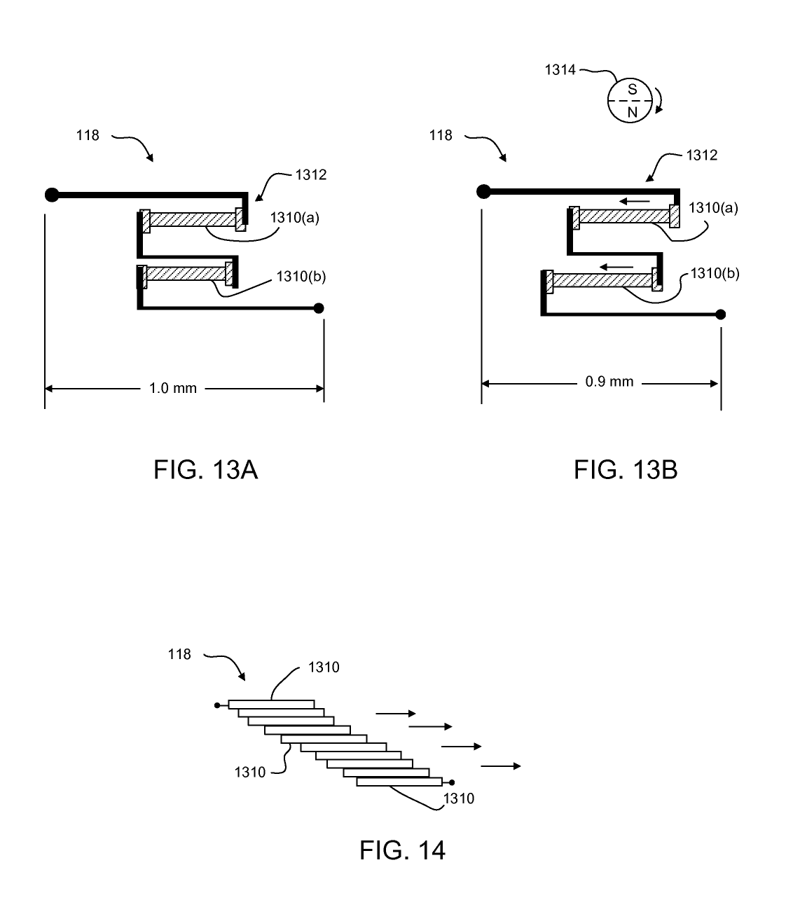

FIGS. 13A and 13B schematically illustrate a tensioning motor 118 comprising “stacked” magnetostrictive elements 1310(a), 1310(b) attached between portions of a rigid or semi-rigid frame 1312 according to one embodiment. The magnetostrictive elements 1310(a), 1310(b) are cylindrical rods or flat plates according to certain embodiments. Other shapes, of course, are also possible. As discussed above, the magnetostrictive elements 1310(a), 1310(b) include Terfenol-D® according to one embodiment. In FIG. 13A, the magnetostrictive elements 1310(a), 1310(b) are connected to each other through the frame 1312 so as to be parallel to one another and are substantially the same length. In this configuration, the tensioning motor 118 has a first overall length (e.g., approximately 1.0 mm in this example embodiment). In FIG. 13B, the magnetostrictive elements 1310(a), 1310(b) are exposed to a magnetic field from, as discussed above, an external magnet 1314. The magnetic field causes the magnetostrictive elements 1310(a), 1310(b) to respectively change their shapes so as to shorten the length of the tensioning motor 118 (e.g., reducing it to approximately 0.9 mm in this example embodiment). An artisan will recognize from the disclosure herein that the tensioning motor's length may also be configured to increase in the presence of the magnetic field.

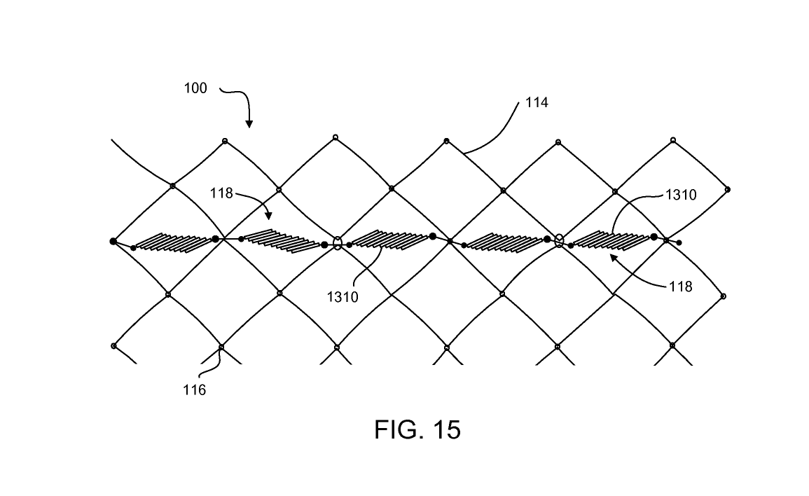

This disclosure is not limited to two magnetostrictive elements 1310(a), 1310(b), as shown in FIGS. 13A and 13B. Rather, the tensioning motor 118 may contain a single magnetostrictive element or any number of magnetostrictive elements, depending on the particular application. For example, FIG. 14 schematically illustrates a tensioning motor 118 having ten magnetostrictive elements 1310. The additional elements 1310 add to the total movement. In one embodiment, for example, dozens of flat magnetostrictive plates may be stacked to magnify the movement induced by the magnetic field.

FIG. 15 schematically illustrates a heart harness 100 that uses the tensioning motor 118 with magnetostrictive elements 1310 shown in FIG. 14 according to one embodiment. The tensioning motors 118 may be oriented and distributed in other configurations, as discussed above. In one embodiment the tensioning motors 118 with magnetostrictive elements 1310 are distributed around the mesh structure of the heart harness 100 so as to squeeze the heart 110 during ventricular contraction (e.g., during a QRS-wave of an electrocardiogram (ECG) signal) and/or to help expand the heart during the relaxation phase of the cardiac cycle (e.g., during the T-wave of the ECG signal). Computerized systems and methods are available for detecting various portions of an ECG signal. Thus, in one embodiment, a magnetic field controlling the magnetostrictive elements 1310 is triggered when a QRS-wave and/or a T-wave of the ECG signal is detected. Accordingly, the heart harness 100 contracts and/or expands in synchronization with the beating of the heart 110. Because Terfenol-D® has a quick response time (e.g., in the microsecond range), the contraction and/or expansion of the magnetostrictive elements 1310 can be synchronized with a human heart rate.

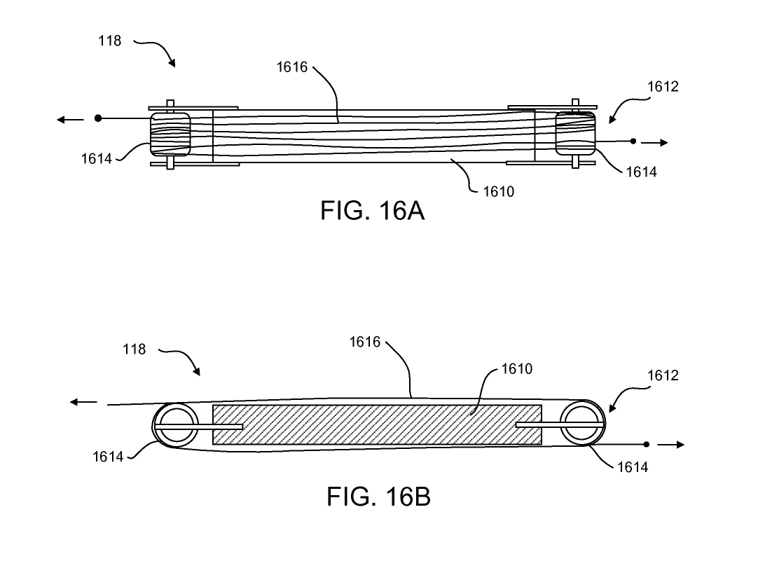

FIGS. 16A and 16B schematically illustrate a tensioning motor 118 that includes a magnetostrictive element 1610 in cooperation with a pulley system 1612 to adjust the tension of the mesh structure of the heart harness 100 discussed above according to one embodiment. FIG. 16A is a top view of the tensioning motor 118 and FIG. 16B is a side view of the tensioning motor 118. The pulley system 1612 includes one or more pulleys 1614 and a wire 1616. Changes in the length of the magnetostrictive elements 1610 are multiplied by the number of pulleys 1614 in the pulley system 1612. In one embodiment, the magnetostrictive element 1610 includes Terfenol-D®.

It will be understood by those having skill in the art that many changes may be made to the details of the above-described embodiments without departing from the underlying principles of the invention. The scope of the present invention should, therefore, be determined only by the following claims.

1. A reversibly adjustable heart harness configured to surround at least a portion of a heart and to provide a compressive force to the heart during at least a portion of a cardiac cycle, the heart harness comprising:
a plurality of wires forming a mesh structure; and
one or more tensioning motors connected to the mesh structure, the one or more tensioning motors configured to selectively increase or reduce tension in the mesh structure to readjust the compressive force provided by the heart harness to the heart.

2. The heart harness of claim 1, wherein the one or more tensioning motors are configured to contract and expand the mesh structure in synchronization with a beating of the heart.

3. The heart harness of claim 1, wherein at least one of the tensioning motors includes a magnetic motor comprising:
a housing; and
a magnet within the housing configured to rotate in the presence of a rotating magnetic field.

4. The heart harness of claim 3, wherein the magnet comprises a cylindrical magnet having magnetic poles divided along a plane running the length of the cylinder.

5. The heart harness of claim 4, wherein at least a portion of the cylindrical magnet is hollow along an axis running the length of the cylinder, and wherein the hollow portion is threaded to engage a portion of the mesh structure so as to pull or push the engaged portion into or out of the hollow portion as the cylindrical magnet rotates.

6. The heart harness of claim 3, wherein the housing comprises a bearing to anchor the rotating magnet.

7. The heart harness of claim 1, wherein at least one of the tensioning motors comprises:
circuitry for counting a number of revolutions of the tensioning motor; and
circuitry for communicating the number of revolutions from within a patient to a receiver located outside the patient.

8. The heart harness of claim 7, wherein the circuitry for communicating comprises a radio frequency identification (RF ID) tag.

9. The heart harness of claim 1, wherein the plurality of wires comprise links between wire segments that deform as the heart expands.

10. The heart harness of claim 1, wherein at least one of the tensioning motors comprises one or more magnetostrictive elements that change shape in response to a magnetic field to adjust the tension in the mesh structure of the heart harness.

11. The heart harness of claim 10, wherein shape change comprises selectively increasing and decreasing a length of the one or more magnetostrictive elements in response to the magnetic field.

12. The heart harness of claim 10, wherein the tensioning motor comprising the one or more magnetostrictive elements further comprises a pulley system.

13. A method for treating a heart with a compressive force during at least a portion of a cardiac cycle, the method comprising:
implanting a reversibly adjustable heart harness around at least a portion of the heart, the heart harness comprising a mesh structure and one or more tensioning motors connected to the mesh structure; and
after implantation, applying an external magnetic field to the one or more tensioning motors to selectively increase or reduce tension in the mesh structure to readjust the compressive force provided by the heart harness to the heart.

14. The method of claim 13, wherein applying the external magnetic field comprises applying a rotating magnetic field.

15. The method of claim 14, wherein applying the rotating magnetic field comprises rotating, outside of a patient's body, a cylindrical magnet having magnetic poles divided along a plane running the length of the cylinder.

16. The method of claim 14, wherein rotating the magnetic field in a first direction increases the tension in the mesh structure and rotating the magnetic field in a second direction reduces the tension in the mesh structure.

17. The method of claim 13, wherein applying the external magnetic field comprises generating an electro-magnetic field with a magnetic resonance imaging (MRI) system.

18. The method of claim 13, further comprising determining an amount of increased or reduced tension in the mesh structure by counting a number of rotations of the external magnetic field with respect to the one or more tensioning motors.

19. The method of claim 13, further comprising increasing and reducing the tension of the mesh structure in synchronization with a beating of the heart.

20. The method of claim 13, further comprising:
determining a number of rotations of each of the tensioning motors; and
communicating the number of rotations from within a patient to a receiver located outside of the patient.
